(19) United States
(12) Patent Application Publication
(10) Pub. No.: US 2021/0349533 A1
(43) Pub. Date: Nov. 11, 2021

(54) INFORMATION PROCESSING METHOD, INFORMATION PROCESSING DEVICE, AND INFORMATION PROCESSING SYSTEM

(71) Applicant: JINS HOLDINGS INC., Maebashi-shi (JP)

(72) Inventor: Hiroki WATANABE, Gunma (JP)

(21) Appl. No.: 17/313,423

(22) Filed: May 6, 2021

(30) Foreign Application Priority Data
May 7, 2020 (JP) 2020081843

(51) Int. Cl. G06F 3/01; G02B 27/00; G02B 27/01

(52) U.S. Cl. CPC G06F 3/013; G02B 27/0093; G02B 2027/0178; G02B 2027/0187; G02B 27/017

(57) ABSTRACT

Usability in interactive character control is improved. An information processing device executed by one or more processors in an information processing device, the information processing method comprising: acquiring at least an electrooculogram signal from another device mounted on a head of a user; detecting at least movement of eyes of a user, based on the electrooculogram signal; display-controlling a character superimposed on an image that is being displayed on a screen; and controlling motion of the character, based on a result of detecting the movement of eyes.

### CROSS-REFERENCE TO RELATED APPLICATIONS

This application claims priority to Japanese Application 2020-081843, filed on May 7, 2020, the content of which is incorporated herein by reference in its entirety.

### BACKGROUND

### Field

The present invention relates to a program, an information processing method, an information processing device, and an information processing system.

### Description of Related Art

Conventionally, there is known control of an avatar superimposed as augmented reality, based on the movement of a user's head detected by a head-mounted display (HMD) mounted on the user's head (e.g., see Japanese Patent No. 6470859 and Patent Publication JP-A-2018-069069).

### SUMMARY

In the conventional technique, an avatar (character) is controlled by using the movement of the head detected by the HMD and in a case of using the movement of a facial part, a camera captures the image of the facial part. This prevents easy superimposing of the avatar from being, making it difficult to provide high usability in interactive character control.

Therefore, a technique disclosed herein aims to improve usability in interactive character control.

A program according to one aspect of the disclosed technique causes an information processing device to execute processing of: acquiring at least an electrooculogram signal from another device mounted on a head of a user; detecting at least movement of eyes of a user, based on the electrooculogram signal; display-controlling a character superimposed on an image that is being displayed on a screen; and performing control on motion of the character, based on a result of detecting the movement of eyes.

According to the disclosed technique, it is possible to improve usability in interactive character control.

### BRIEF DESCRIPTION OF DRAWINGS

FIG. 1 is a diagram illustrating an example of an information processing system according to an embodiment;

FIG. 2 is a schematic block diagram illustrating a hardware configuration of an information processing device according to the embodiment;

FIG. 3 is a block diagram illustrating an example of a configuration of a processing device according to the embodiment;

FIG. 4 is a diagram illustrating an example of a configuration of the information processing device according to the embodiment;

FIG. 5 is a diagram illustrating Example 1 of a display screen of an application A in the embodiment;

FIG. 6 is a diagram illustrating Example 2 of a display screen of the application A in the embodiment;

FIG. 7 is a diagram illustrating Example 3 of a display screen of the application A in the embodiment;

FIG. 8 is a diagram illustrating Example 4 of a display screen of the application A in the embodiment;

FIG. 9 is a diagram illustrating Example 5 of a display screen of the application A in the embodiment;

FIG. 10 is a diagram illustrating Example 6 of a display screen of the application A in the embodiment; and

FIG. 11 is a sequence diagram illustrating an example of processing for the application A in the embodiment.

### DETAILED DESCRIPTION

An embodiment of the present invention will be described below with reference to the drawings. Note that the embodiment described below is merely an example, and there is no intention to exclude the application of various modifications and techniques not explicitly described below. In other words, the present invention can be implemented with various modifications without departing from the scope and spirit of the invention. In the following description for the drawings, the same or similar parts are denoted by the same or similar reference numerals. The drawings are schematic and do not necessarily correspond to actual dimensions, ratios, and the like. Between mutual drawings, differences in relation of their dimensions and ratios may also be included.

### EMBODIMENT

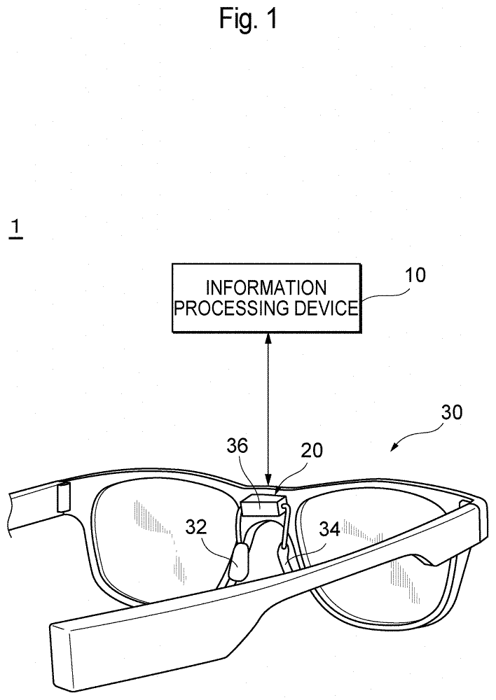

In the embodiment, an eyewear is taken as an example of a wearable terminal including an acceleration sensor, an angular velocity sensor, and a bioelectrode, but the present invention is not limited to this. FIG. 1 is a diagram illustrating an example of an information processing system 1 according to the embodiment. The information processing system 1 illustrated in FIG. 1 includes an information processing device 10 and an eyewear 30. The information processing device 10 and the eyewear 30 are connected to each other via a network to enable data communication.

The eyewear 30 includes a processing device 20 on, for example, its bridge part. The processing device 20 includes bioelectrodes 32, 34, and 36 that are arranged on a pair of nose pads and the bridge part, respectively. The processing device 20 may include a 3-axis acceleration sensor and a 3-axis angular velocity sensor, which may be a 6-axis sensor.

The processing device 20 detects a sensor signal, an electrooculogram signal, and the like and transmits the detected signals to the information processing device 10. The position where the processing device 20 is installed does not necessarily have to be in the bridge part, and may be determined to be any position as long as the electrooculogram signal can be acquired in a state where the eyewear 30 is worn. The processing device 20 may also be detachably provided on the bridge part.

The information processing device 10 is an information processing device having a communication function. For example, the information processing device 10 is preferably a mobile terminal such as a smartphone owned by a user, or is a personal computer, a tablet terminal, or the like. The information processing device 10 detects the movement of the eyes of the user and the movement of the head of the user based on the electrooculogram signal, the sensor signal, and the like received from the processing device 20, and controls a character (e.g., avatar) to be superimposed on an image being displayed on a screen based on the result of the detection.

Hardware Configuration of Information Processing Device 10

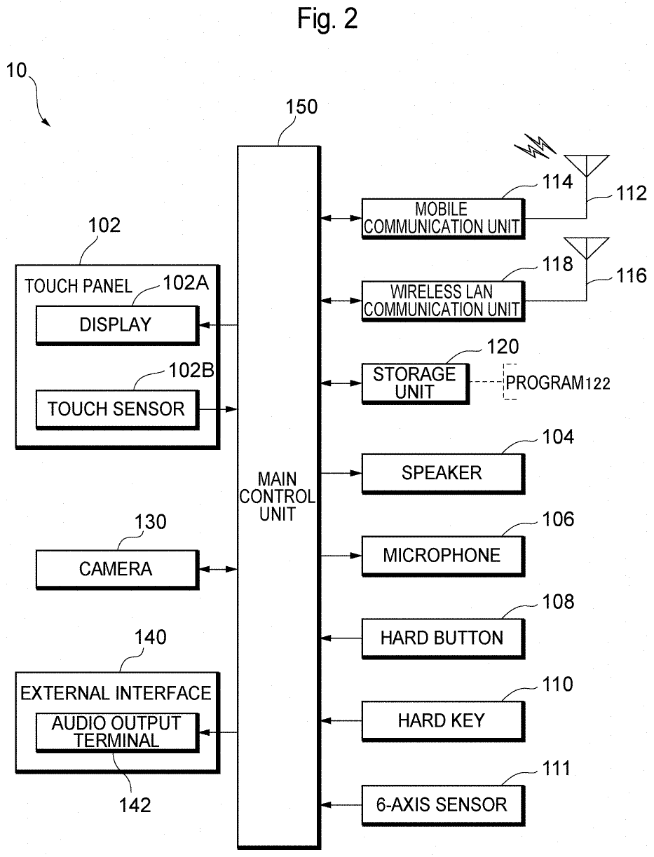

FIG. 2 is a schematic block diagram illustrating a hardware configuration of the information processing device 10 according to the embodiment. A typical example of the information processing device 10 is a mobile terminal such as a smartphone. Other examples of the information processing device 10 according to the embodiment can include general-purpose devices capable of displaying a screen while processing data through network communication, such as a mobile terminal capable of wirelessly or wired connection to a network, and an electronic device equipped with a touch panel such as a tablet terminal.

The information processing device 10 according to the embodiment has, for example, a rectangular, low-profile housing (not illustrated), and includes a touch panel 102 configured on one surface of the housing. In the information processing device 10, multiple components are connected to a main control unit 150. The main control unit 150 is, for example, one or more processors.

A mobile communication antenna 112, a mobile communication unit 114, a wireless LAN communication antenna 116, a wireless LAN communication unit 118, a storage unit 120, a speaker 104, a microphone 106, a hard button 108, a hard key 110, and a 6-axis sensor 111 are connected to the main control unit 150. In addition, the touch panel 102, a camera 130, and an external interface 140 are connected to the main control unit 150. The external interface 140 includes an audio output terminal 142.

The touch panel 102 has both a function of a display device and a function of an input device, and includes a display (display screen) 102A having a display function and a touch sensor 1026 having an input function. The display 102A is, for example, a general display device such as a liquid crystal display or an organic electro luminescence (EL) display. The touch sensor 102B is configured to include elements for detecting a contact operation which are arranged on the front surface of the display 102A and a transparent operation film which is laminated on the elements. As the contact detection method of the touch sensor 1026, any of known methods such as a capacitance type, a resistive film type (pressure sensitive type), and an electromagnetic induction type can be adopted.

The touch panel 102 serving as a display device displays an application image generated by the main control unit 150 executing a program 122. The touch panel 102 serving as an input device detects the action of a contact object that comes into contact with the surface of the operation film to receive an operation input, and then sends information on its contact position to the main control unit 150. Note that examples of the contact object include a player's finger and a stylus, and a finger or fingers are used herein as a typical example. The action of the finger(s) is detected as coordinate information indicating the position(s) or region of the contact point, and the coordinate information represents, for example, coordinate values on two axes which extend in the short side direction and the long side direction of the touch panel 102.

The storage unit 120 stores the program 122 that executes processing related to a character to be superimposed on a displayed image. The storage unit 120 may be separate from the information processing device 10, and may be, for example, a recording medium such as an SD card or a CD-RAM, or a non-transitory recording medium.

The information processing device 10 is connected to a network N through the mobile communication antenna 112 and the wireless LAN communication antenna 116 to perform data communication with the processing device 20.

Configuration of Processing Device 20

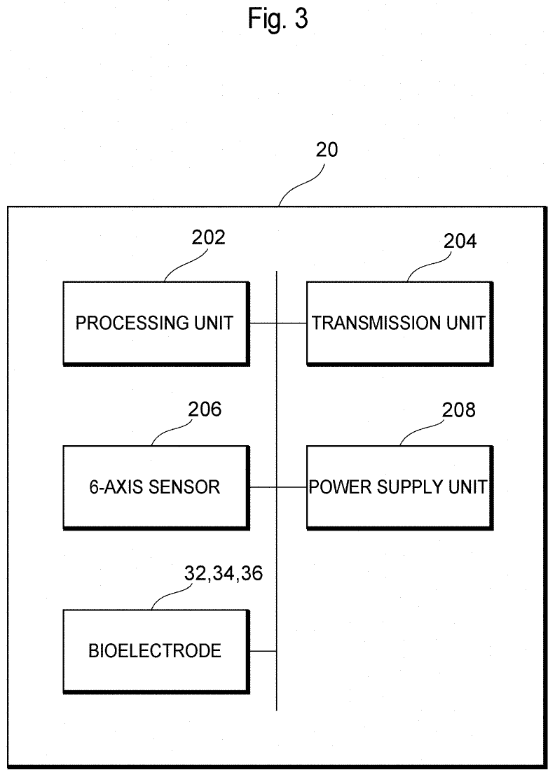

FIG. 3 is a block diagram illustrating an example of a configuration of the processing device 20 according to the embodiment. As illustrated in FIG. 3, the processing device 20 includes a processing unit 202, a transmission unit 204, a 6-axis sensor 206, a power supply unit 208, and the bioelectrodes 32, 34, and 36. Further, the bioelectrodes 32, 34, and 36 are connected to the processing unit 202 by using an electric wire, for example, via an amplification unit.

The 6-axis sensor 206 is a 3-axis acceleration sensor and a 3-axis angular velocity sensor. Each of these sensors may be provided separately. The 6-axis sensor 206 outputs a detected sensor signal to the processing unit 202.

The processing unit 202 processes the sensor signal obtained from the 6-axis sensor 206 and the electrooculogram signal obtained from each of the bioelectrodes 32, 34, and 36 as necessary, and for example, packetizes the sensor signal and the electrooculogram signal, and outputs the resulting packet to the transmission unit 204. The processing unit 202 also includes a processor, and for example, the processing unit 202 may use the electrooculogram signal to calculate first biological information regarding eye blink and second biological information regarding the movement of the line of sight.

The processing unit 202 may use the sensor signal from the 6-axis sensor 206 to calculate third biological information regarding the movement of the head. The information regarding the movement of the head is, for example, information regarding the movement of the head back, forth, left and right. The processing unit 202 may only amplify the sensor signal obtained from the 6-axis sensor 206. Hereinafter, the processing unit 202 will be described as performing processing of packetizing the electrooculogram signal and the sensor signal.

The transmission unit 204 transmits the electrooculogram signal and/or sensor signal packetized by the processing unit 202 to the information processing device 10. For example, the transmission unit 204 transmits the electrooculogram signal and/or the sensor signal to the information processing device 10 by wireless communication such as Bluetooth (registered trademark) and wireless LAN, or wired communication. The power supply unit 208 supplies electric power to the processing unit 202, the transmission unit 204, the 6-axis sensor 206, and so on.

Configuration of Information Processing Device 10

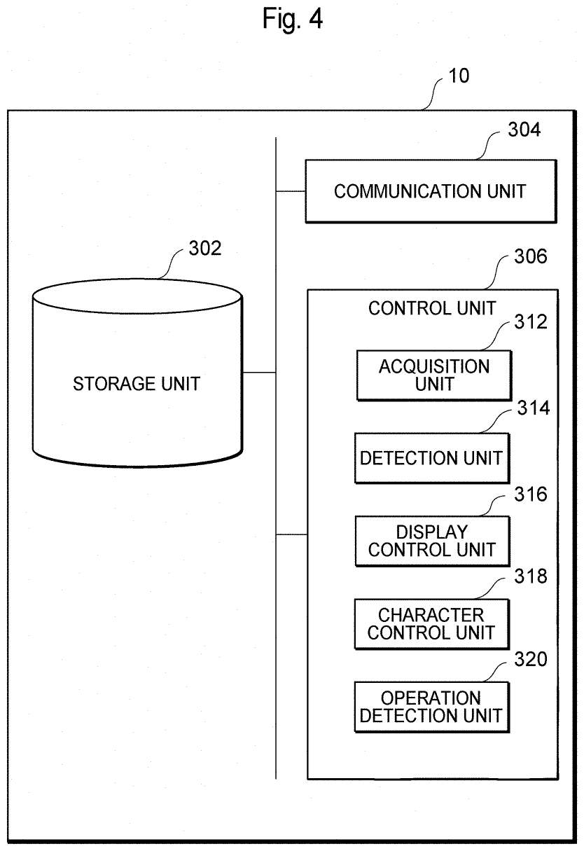

Next, a configuration of the information processing device 10 will be described. FIG. 4 is a diagram illustrating an example of the configuration of the information processing device 10 according to the embodiment. The information processing device 10 includes a storage unit 302, a communication unit 304, and a control unit 306.

The storage unit 302 can be realized by, for example, the storage unit 120 illustrated in FIG. 2. As an example, the storage unit 302 stores data and the like related to an application (hereinafter, also referred to as the application A) that executes processing for generating an image on which a character is superimposed using augmented reality (AR) technology. The data related to the application A is, for example, data received from the processing device 20, information related to the display and control of the character, image data on which the character is superimposed, screen information to be displayed on the screen, and the like. The character includes, for example, an avatar, and the image includes a still image or a video.

The communication unit 304 can be realized by, for example, the mobile communication unit 114, the wireless LAN communication unit 118, and/or the like. The communication unit 304 receives data from, for example, the processing device 20. The communication unit 304 may transmit the data processed by the information processing device 10 to a server. In other words, the communication unit 304 has functions as a transmission unit and a reception unit.

The control unit 306 can be realized by, for example, the main control unit 150 or the like. The control unit 306 executes the application A. The application A in the embodiment acquires the electrooculogram signal and/or the sensor signal, detects the movement of the user's eyes and the movement of the user's head based on the respective signals, and controls the motion of the character based on the detection result. The control unit 306 also superimposes the character to be controlled on an image, generates an image including the character, and saves the generated image. In order to implement this function, the control unit 306 includes an acquisition unit 312, a detection unit 314, a display control unit 316, a character control unit 318, and an operation detection unit 320.

The acquisition unit 312 acquires the signal received by the communication unit 304. For example, the acquisition unit 312 acquires at least an electrooculogram signal from another device (e.g., the eyewear 30) worn on the user's head.

The detection unit 314 detects at least the movement of the user's eyes based on the electrooculogram signal acquired by the acquisition unit 312. For example, the detection unit 314 detects the movement of the eyes including eye blink and the movement of the line of sight based on the electrooculogram signal by a known technique.

The display control unit 316 performs display control in which a character is superimposed on an image being displayed on the screen (the display 102A) of the information processing device 10. The image may be an image selected by the user or an image being captured by the camera 130.

The character control unit 318 controls the motion of the character based on the result of detecting the movement of the eyes by the detection unit 314 and/or a command described later. For example, the character control unit 318 controls the eye blink and movement of the eyes of the character superimposed on the image in synchronization with the detected eye blink and movement of the eyes, respectively. The character may be, for example, a preset avatar or an avatar selected by the user.

The character control unit 318 has a plurality of motion parameters for controlling the movement of the character, and may associate the eye blink, the movement of the line of sight, and the movement of the head of the user with the motion parameters for the character. This association may be made in accordance with a user operation. The plurality of motion parameters include, for example, parameters related to the character's eye blink, parameters related to the movement of the line of sight, parameters related to the movement of the head, parameters related to the movement of the torso, parameters related to zoom-out and zoom-in of the character, and parameters related to the movement of the character's hand(s), and the like.

The operation detection unit 320 detects a user operation on a UI component displayed on the screen, and outputs various commands to the corresponding unit in response to the user operation. For example, in response to detecting an operation such as a character selection button, a character basic motion button, and a character facial expression button, the operation detection unit 320 outputs a command corresponding to the detected operation to the character control unit 318.

As described above, it is possible to improve usability in interactive character control based on the electrooculogram signal acquired from the user who directs the control of the character without capturing an image of the user's face with the camera. For example, based on the electrooculogram signal, various controls become possible, and the movement of the eyes of the character and the like can be controlled more precisely.

The acquisition unit 312 may acquire a sensor signal sensed by the acceleration sensor and/or the angular velocity sensor included in another device (e.g., the eyewear 30). In this case, the detection unit 314 may detect the movement of the user's head based on the sensor signal. The character control unit 318 may control the motion of the character based on the result of detecting the movement of the user's eyes and the movement of the user's head.

Controlling the motion of the character includes, for example, controlling the character's eye blink and the movement of the character's line of sight in synchronization with the user's eye blink and the movement of the user's line of sight, and controlling the movement of the character's head in synchronization with the movement of the user's head. Controlling the motion of the character may include, for example, determining motion parameters for controlling a predetermined motion A of the character based on the user's eye blink and the movement of the user's line of sight, and determining motion parameters for controlling a predetermined movement B of the character based on the movement of the user's head.

This makes it possible to improve usability in interactive character control also based on the movement of the head of the user who directs the control of the character. For example, it is possible to increase the variation of motions to be controlled based on the electrooculogram signal and the movement of the head.

The display control unit 316 may control the display of a UI component for selecting a basic motion related to the character's torso on the screen of the information processing device 10. In this case, the character control unit 318 may control the motion of the character based on the basic motion related to the character's torso selected by the user using the UI component and a motion related to the character's head according to the detection result.

For example, the display control unit 316 controls so that a selection button is displayed on the screen for allowing the user to select a preset basic motion related to the character's torso. The character control unit 318 controls so that the basic motion in accordance with a command corresponding to the button selected by the operation detection unit 320 is reflected in the basic motion of the torso of the character being displayed.

This makes it possible to control the motion of the character to be divided into the motion of the head and the motion of the torso so that the motion of the head is controlled according to the movement of the user's eyes while the motion of the torso is easily controlled using the UI component displayed on the screen. As a result, it is possible to improve usability in interactive character control.

As described above, using the eyewear 30 provided with a biometric information measurement system makes it possible to separate the functions of operating the avatar's head and operating the avatar's torso. Accordingly, displaying a character's torso operation interface on the screen makes it possible for the user to operate the character intuitively without the need for cooperation with an expensive external wearable terminal even in complicated and complex operations.

The display control unit 316 may perform display control in which the character is superimposed on an image being captured by an image capturing device (e.g., the camera 130). For example, the camera 130 may be activated by the user, the display control unit 316 may superimpose the character on an image being captured by using AR technology, the character control unit 318 may control the motion of the superposed character, and the control unit 306 may save an image including the superimposed character.

This results in no need to capture the movement of the user's eyes and the like with the camera 130, so that the camera 130 can capture an image on which the character is to be superimposed, and easily implement capturing the image in real time, and superimposing and controlling the character. For example, it is possible to interactively control a character being superimposed while capturing a video with a rear camera of a mobile terminal.

The detection unit 314 may detect eye blink or the movement of the line of sight based on the electrooculogram signal. As a method for detecting the eye blink or the movement of the line of sight, a known method can be used. In this case, the character control unit 318 may control the motion of the character based on a first motion parameter associated with the eye blink or a second motion parameter associated with the movement of the line of sight. For example, the character control unit 318 may control so that the user's eye blink or the movement of the user's line of sight is reflected in the character's eye blink (the first motion parameter) or the movement of the character's line of sight (the second motion parameter) in real time. The character control unit 318 may control so that the user's eye blink or the movement of the user's line of sight is associated with two other motion parameters for the character. For example, the user's eye blink or the movement of the user's line of sight may be associated with a first motion parameter or a second motion parameter related to the motion of the character's torso.

This makes it possible to improve usability in interactive character control by using a detection result acquired from an electrooculogram signal such as of eye blink or the movement of the line of sight. In addition, it is possible to increase the variation of motions to be controlled.

Further, the detection unit 314 may detect the strength of the eye blink based on the electrooculogram signal or the speed of the movement of the line of the sight. For example, based on the electrooculogram signal, the detection unit 314 may set a plurality of threshold values for a signal strength to detect the strength as one of a plurality of levels into which eye blink is divided, and for the speed of the movement of the line of sight, the detection unit 314 may set a plurality of threshold values for a horizontal movement speed to detect the speed as one of a plurality of levels into which movement of line of sight is divided.

In this case, the character control unit 318 may control the motion of the character based on a third motion parameter associated with the strength of the eye blink or a fourth motion parameter associated with the speed of the movement of the line of sight. Which motion parameter is associated with each of the strength of the eye blink and the speed of the movement of the line of sight may be preset by the user. For example, the character control unit 318 may change the magnitude of the motion of the character according to the strength of the eye blink. As an example, the higher the strength of the eye blink, the wider the character swings the hand. The character control unit 318 may also change the speed of the motion of the character according to, for example, the speed of the movement of the line of sight. As an example, the faster the movement of the line of sight, the faster the cycle of swaying of the character.

This makes it possible to improve usability in interactive character control by using a parameter peculiar to the electrooculogram signal such as of the strength of the eye blink or the speed of the movement of the line of sight. In addition, it is possible to increase the variation of motions to be controlled.

The character control unit 318 may change the position of the character in the depth direction with respect to the screen according to the movement of the head included in the detection result. For example, the character control unit 318 sets a virtual camera at a predetermined position in front of the screen of the information processing device 10 (on the user side from which the screen is viewed), controls so that the character moves closer to the virtual camera and the size of the character on the screen becomes larger when the user tilts the head forward, and controls so that the character moves away from the virtual camera and the size of the character on the screen becomes smaller when the user tilts the head backward.

Note that a part of the character to be controlled is the entire character to be displayed, the part above the torso, or the head, and any one of them may be determined in advance, and the number of parts of the character to be controlled may increase depending on the degree of tilt of the user's head. For example, as the tilt of the user's head increases, the number of parts of the character to be controlled may increase in the order of the head, the part above the torso, and the entire character to be displayed.

This makes it possible to control the perspective and size of the character by using the movement of the user's head and thus improve usability in interactive character control.

The motion of the character may include an active motion in which the character automatically takes a motion such that a predetermined motion is repeatedly performed for a predetermined period when predetermined directions or instructions are received, and a passive motion in which the character takes a motion each time directions are received. The active motion includes, for example, a motion of repeating a constant motion such as swaying once the active motion is set by the user. The passive motion includes, for example, a motion in which a predetermined motion such as a greeting or a surprise gesture is performed when predetermined directions or instructions are received and any motion is not performed until the next directions or instructions are received.

In this case, the character control unit 318 may determine the value of a parameter related to the active motion based on the detection result. For example, for an active motion of swaying, the character control unit 318 may determine a parameter related to a swaying cycle based on the number of eye blinks or the like for a predetermined period.

This makes it possible to reflect a user's unconscious motion on the character by using the movement of the user's eyes or the movement of the user's head even when the character automatically takes a motion, and thus improve usability in interactive character control.

The detection unit 314 may detect the degree of concentration or the degree of calmness of the user by using a known technique based on the eye blink detected from the electrooculogram signal. The known technique is, for example, the technique described in Patent Publication JP-A-2017-70602 from the same applicant, which describes that the degree of concentration and the degree of calmness are detected by using eye blink or the movement of the line of sight.

The character control unit 318 may control the motion of the character based on the degree of concentration or the degree of calmness included in the detection result. For example, as an example of character motion control, the character control unit 318 may change the facial expression of the character to a facial expression of concentration when it is determined that the user is concentrated, change the facial expression of the character to a relaxed facial expression when it is determined that the user is calm, or change the complexion of the character and the brightness and saturation of the screen including the character according to the user's concentration or calmness.

This makes it possible to reflect the user's psychological state on the character by using a psychological state (concentration, calmness, etc.) that the user is not very aware of, and thus improve the smoothness of the motion of the character in interactive character control.

The information processing device 10 may be a mobile terminal such as a smartphone which is an example of a mobile processing terminal, and the other device may be the eyewear 30 which is an example of a wearable terminal.

Since such a mobile terminal is usually owned by the user, which is highly portable and versatile, it is possible for the mobile terminal to move while sensing the electrooculogram signal and the like and thus increase the variation of videos on which the character is to be superimposed. For example, it is possible to interactively control a character being superimposed while capturing a video with a rear camera of a mobile terminal.

The character control unit 318 may change the character to another character by using the movement of the line of sight. For example, the character control unit 318 sets a threshold value for the amount of movement of the line of sight, and changes the character being displayed on the screen when the amount of movement is equal to or greater than the threshold value. Note that, if a character change button is pressed by the user in advance, the character control 318 may start processing of changing the character by moving the line of sight. In this way, the character control unit 318 may associate the movement of the user's eyes with various operations related to the character.

This association of the movement of the user's eyes with various operations related to the character makes it possible to improve usability in character control.

Screen Example

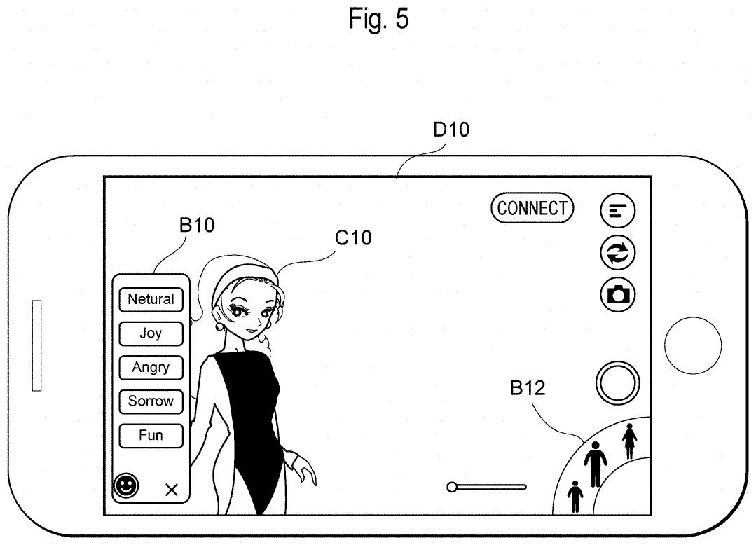

Next, a screen example of the application A in the embodiment will be described with reference to FIGS. 5 to 10. FIG. 5 is a diagram illustrating Example 1 of a display screen of the application A in the embodiment. On a screen D10 illustrated in FIG. 5, for example, selection buttons B10 for face actions (e.g., 5 types) are displayed on the left, and a selection revolver B12 for body actions (e.g., 27 types) is displayed on the right. The operation detection unit 320 detects an operation on this screen, and then the character control unit 318 determines a basic facial expression (basic motion) of the face of a character C10 and determines a basic motion related to the torso of the character C10.

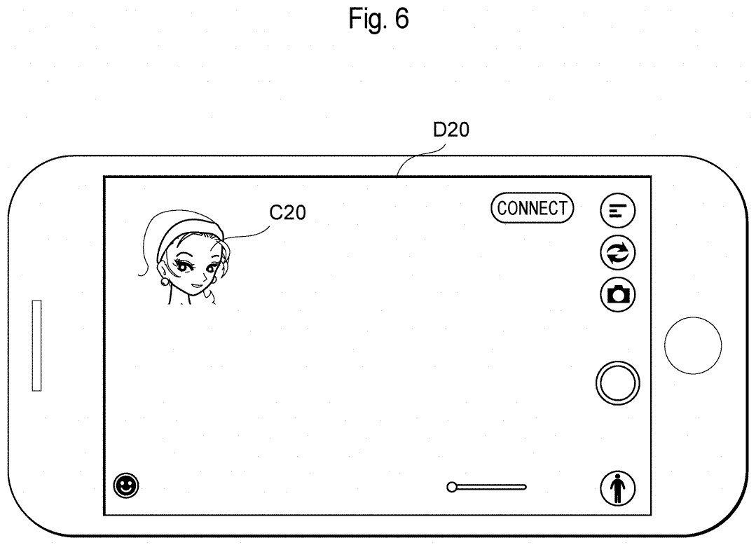

FIG. 6 is a diagram illustrating Example 2 of a display screen of the application A in the embodiment. A screen D20 illustrated in FIG. 6 is an example of a screen in which a character C20 which is a cut-out of the character C10 is displayed on the upper left in response to tapping of a cut-out button. At this time, even in the cut-out, the face and body actions can be performed by the character control unit 318.

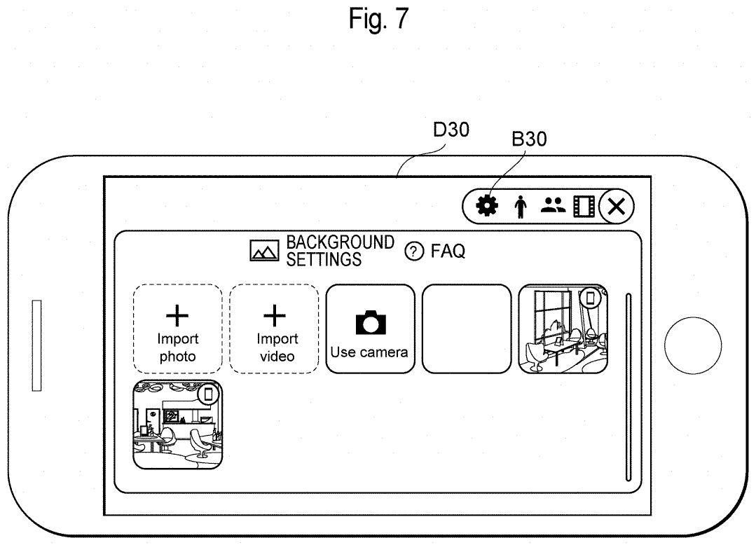

FIG. 7 is a diagram illustrating Example 3 of a display screen of the application A in the embodiment. A screen D30 illustrated in FIG. 7 is an example of a screen when a button area is displayed on the upper right and then a gear button B30 is tapped in the button area. Tapping the gear button B30 makes it possible to select an image on which the character is to be superimposed.

For example, the user taps “Import photo” to import a still image, and taps “Import video” to import a video. The user also taps “Use camera” to acquire a video or the like with the rear camera. Note that a white background may be included in the initial settings for items to be selected. A captured photo and video are automatically listed up on the screen D30, so that the user can select an image/video to be used by tapping.

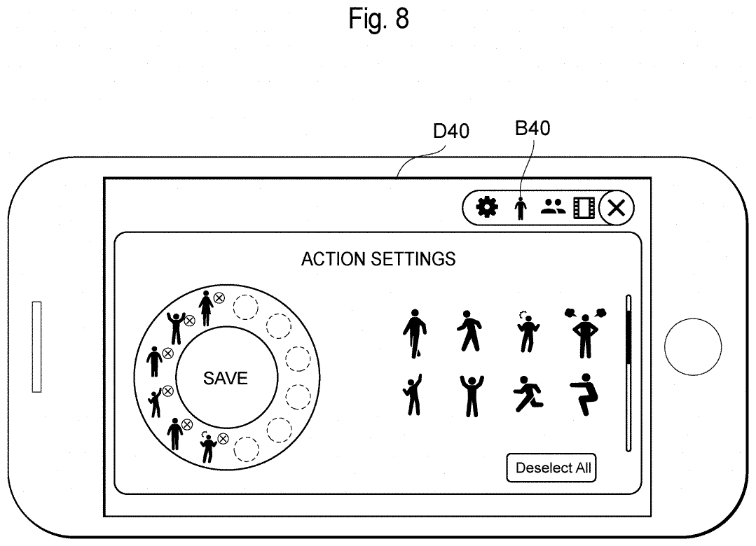

FIG. 8 is a diagram illustrating Example 4 of a display screen of the application A in the embodiment. A screen D40 illustrated in FIG. 8 is an example of a screen when a button area is displayed on the upper right and then a person-shaped button B40 is tapped in the button area. In this example, tapping the person-shaped button B40 makes it possible to select a basic motion related to the character's torso. For example, a total of 27 types of basic motions related to the torso (body) are prepared. For example, when the user selects 12 types from among the 27 types, the 12 types of basic motions are added to the selection revolver B12 illustrated in FIG. 5, so that the 12 types can be selectable.

Note that, as described above, the basic motion (action) may include two types: “active motions” and “passive motions”. The “active motions” include “swaying”, “marching”, “running”, “default sitting”, “playing a game”, “air chair”, “eating meal”, “standing up”, and so on. The “passive motions” basically include motions other than the “active motions”.

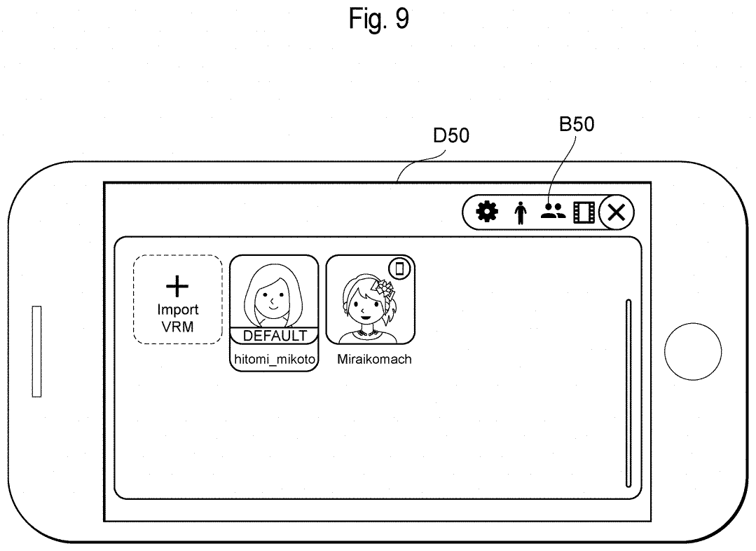

FIG. 9 is a diagram illustrating Example 5 of a display screen of the application A in the embodiment. A screen D50 illustrated in FIG. 9 is an example of a screen when a button area is displayed on the upper right and then a model button B50 is tapped in the button area. In this example, tapping the model button B50 makes it possible to select a model of the character.

The model of the character includes a default model preset in this application A, a model created from a VRM file for a 3D avatar, and the like. The user can import a predetermined VRM file into this application A in advance. If the VRM file has been imported, the user can tap “Import VRM” to use the VRM file.

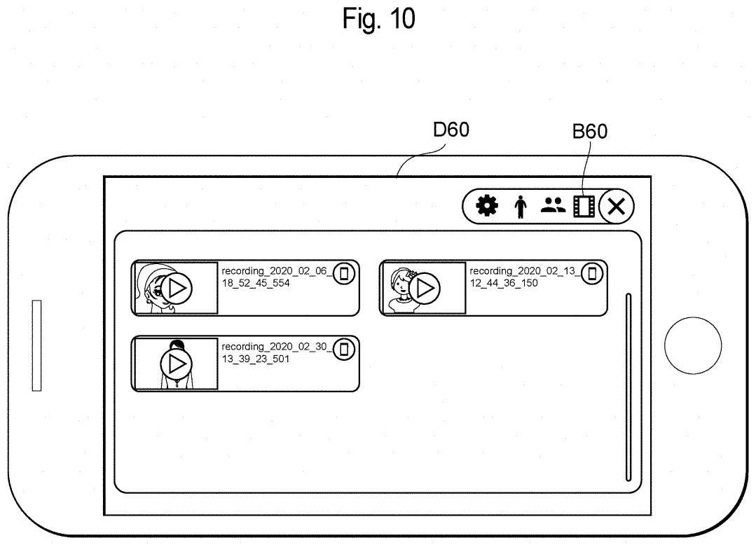

FIG. 10 is a diagram illustrating Example 6 of a display screen of the application A in the embodiment. A screen D60 illustrated in FIG. 10 is an example of a screen when a button area is displayed on the upper right and then a film button B60 is tapped in the button area. In this example, tapping the film button B60 makes it possible to select image data on which the character created by the user is superimposed and browse it. Images such as captured videos and photographs (still images) may be simultaneously saved in a photo application of the information processing device 10. Note that an image file on which the character is superimposed is output in the MP4 file format, so that it can be easily taken out, shared on SNS or the like, and processed.

Operation

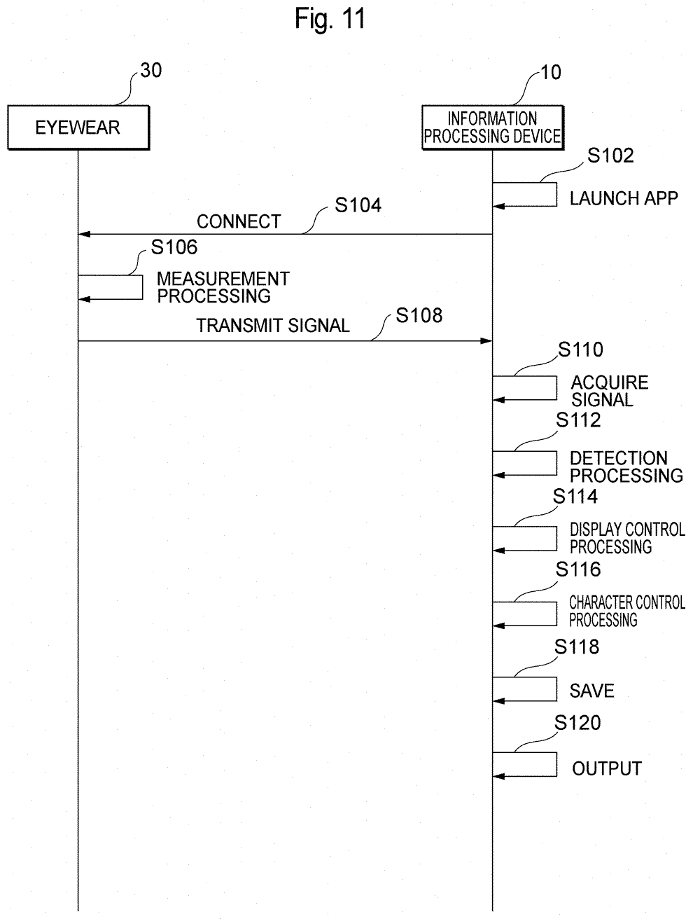

Next, steps of processing in an information processing system 1 in the embodiment will be described. FIG. 11 is a sequence diagram illustrating an example of processing for the application A in the embodiment.

In step S102 illustrated in FIG. 11, the control unit 306 launches the application A in response to a user operation.

In step S104, the control unit 306 establishes communication with the eyewear 30. The communication is, for example, Bluetooth (registered trademark) or Wi-fi (registered trademark).

In step S106, the processing device 20 of the eyewear 30 measures an electrooculogram signal from the user with the bioelectrodes 32, 34, and 36. In a case where the processing device 20 includes an acceleration sensor/angular velocity sensor, these sensors are also used for measurement.

In step S108, the processing device 20 of the eyewear 30 transmits the acquired electrooculogram signal and/or sensor signals to the information processing device 10.

In step S110, the acquisition unit 312 of the information processing device 10 acquires the electrooculogram signal transmitted from the eyewear 30 worn on the user's head. The acquisition unit 312 may also acquire the sensor signals indicating the movement of the user's head.

In step S112, the detection unit 314 of the information processing device 10 detects the movement of the user's eyes based on the acquired electrooculogram signal. The detection unit 314 may detect eye blink or the movement of the line of sight. When a sensor signal is acquired, the detection unit 314 may detect the movement of the head (front-back direction with respect to the face, lateral direction with respect to the face, up and down direction with respect to the face), which are included in the detection result.

In step S114, the display control unit 316 performs display control in which the character is superimposed on an image being displayed on the screen. Note that the image to be displayed may be selected by the user.

In step S116, the character control unit 318 controls the motion of the character superimposed on the image and displayed based on the result of detecting the movement of the user's eyes. When the detection result includes the movement of the user's head, the character control unit 318 may control the motion of the character according to the movement of the head.

In step S118, the control unit 306 stores image data including the motion of the superimposed character in the storage unit 120 of the information processing device 10. For example, the image data is saved in the MP4 format, but the format is not limited to this, and the image data can be saved in a file format suitable for the purpose.

In step S120, the control unit 306 outputs the saved image data including the superimposed character to an external device or the like to upload it to SNS or attach it to an e-mail.

Note that the processing steps included in the processing flow described with reference to FIG. 11 can be executed in any order or in parallel as long as there is caused no contradiction in the processing content, and additional step(s) may also be inserted between processing steps. The step referred to as one step for convenience can be divided into a plurality of steps to be executed, while the steps referred to as separate steps for convenience can be regarded as one step.

As described above, according to the embodiment, it is possible to improve usability in interactive character control. In a case where a 3D model is controlled, according to the embodiment, by importing a registered humanoid VRM data model, the character (e.g., an avatar) desired by the user can be superimposed on the image.

The user can also move his/her eyes and head to control the character superimposed on the image. According to the embodiment, the user can operate the actions of the face and body of the character as if the user operated a game controller (see, for example, FIGS. 5 to 10).

In respect to the superimposed image, it is possible for the user to capture an AR composite video and capture a still image with the rear camera of a mobile terminal, and the like. In addition, it is possible for the user to operate a character (e.g., an avatar) which is AR-synthesized with a video or a still image that has already been captured to serve as a background.

According to the character control described above, transferring a part of the control to the eyewear 30 equipped with the biometric information measurement system makes it possible for the user to allow intuitive operation without using a camera and carry a device sensing and capturing images.

Using the eyewear 30 equipped with the biometric information measurement system makes it possible to perform AR synthesis through a UI that makes it easy to edit without requiring editing literacy for the application A of the information processing device 10.

As described above, it is possible to capture an image with the rear camera of the information processing device 10, and it is possible to control a character that can be intuitively controlled while capturing an image at the time of AR synthesis.

In addition, it is possible to provide an interactive character control application for image capturing using AR in third-person point of view, instead of the conventional character capturing application for viewing using AR.

Note that, in the embodiment, a case where the eyewear 30 is glasses has been described. However, the eyewear is not limited to this. The eyewear may be any eye-related wearables, and may be on-face wearables or head wearables such as glasses, sunglasses, goggles, a head-mounted display, and a frame thereof.

Note that, in the embodiment, the use of sensor signals from the 6-axis sensor included in the eyewear 30 has been described, but also in a case of using sensor signals from the 6-axis sensor 111 included in the information processing device 10, it is possible to execute the application described in the embodiment. In other words, the 6-axis sensor may be mounted not only to the head but also to any position on the human body.

Although the present invention has been described above with reference to the embodiment, the technical scope of the present invention is not limited to the scope described in the above embodiment. It would be apparent to those skilled in the art that various modifications or improvements can be made to the above embodiment. It would be clear from the claims that such modifications or improvements may also be included in the technical scope of the present invention.

What is claimed is:

1. An information processing method to be executed by one or more processors included in an information processing device, the information processing method comprising:
acquiring at least an electrooculogram signal from another device mounted on a head of a user;
detecting at least movement of eyes of a user, based on the electrooculogram signal;
display-controlling a character superimposed on an image that is being displayed on a screen; and
controlling motion of the character, based on a result of detecting the movement of eyes.

2. The information processing method according to claim 1, wherein
the acquiring includes acquiring a sensor signal sensed by an acceleration sensor and/or an angular velocity sensor mounted on the other device,
the detecting includes detecting the movement of the head of the user, based on the sensor signal, and
the controlling the motion includes controlling the motion of the character, based on the result of detecting the movement of the eyes and the movement of the head.

3. The information processing method according to claim 1, wherein
the display-controlling includes display-controlling a UI component, which is selectable of a basic motion relating to a torso of the character, to be displayed on the screen, and
the controlling the motion includes controlling the motion of the character, based on the basic motion relating to the torso of the character selected using the UI component and a motion relating to the head of the character, based on the result of detecting.

4. The information processing method according to claim 1, wherein
the display-controlling includes display-control of the character superimposed on an image that is being captured by an image capturing device.

5. The information processing method according to claim 1, wherein
the detecting includes detecting eye blink or movement of line of sight, based on the electrooculogram signal,
the controlling the motion includes controlling the motion of the character, based on a first motion parameter associated with the eye blink or a second motion parameter associated with the movement of line of sight.

6. The information processing method according to any one of claim 1, wherein
the detecting includes detecting strength of eye blink or speed of movement of line of sight, based on the electrooculogram signal,
the controlling the motion includes controlling the motion of the character, based on a third motion parameter associated with the strength of eye blink or a fourth motion parameter associated with the speed of movement of line of sight.

7. The information processing method according to claim 2, wherein
the controlling the motion includes changing a position of the character in a depth direction with respect to the screen according to the movement of the head included in the result of detecting.

8. The information processing method according to claim 1, wherein
the motion of the character includes an active motion that is an automatic motion, and
the controlling the motion includes determining a value of a parameter relating to the active motion, based on the result of detecting.

9. The information processing method according to claim 1, wherein
the detecting includes detecting a degree of concentration or a degree of calmness of the user by using eye blink detected based on the electrooculogram signal, and
the controlling the motion includes controlling the motion of the character, based on the degree of concentration or the degree of calmness included in the result of detecting.

10. The information processing method according to claim 1, wherein
the information processing device is a mobile terminal, and the other device is an eyewear.

11. An information processing device comprising one or more processors,
the one or more processors executing processing including:
acquiring at least an electrooculogram signal from another device mounted on a head of a user;
detecting at least movement of eyes of a user, based on the electrooculogram signal;
display-controlling of a character superimposed on an image that is being displayed on a screen; and
controlling motion of the character, based on a result of detecting the movement of eyes.

12. An information processing system comprising an information processing device and an eyewear connected to be able to implement data communication, wherein
the eyewear includes
a plurality of bioelectrodes, and
a transmission unit configured to transmit an electrooculogram signal acquired from the plurality of bioelectrodes to the information processing device, and
the information processing device includes
a communication unit configured to receive at least the electrooculogram signal from the eyewear, and
a control unit configured to detect at least movement of eyes of a user, based on the electrooculogram signal, display-control a character superimposed on an image that is being displayed on a screen, and control motion of the character, based on a result of detecting the movement of eyes.
